(19) United States
(12) Patent Application Publication
(10) Pub. No.: US 2006/0171980 A1
(43) Pub. Date: Aug. 3, 2006

(54) Implantable or insertable medical devices having optimal surface energy

(76) Inventors: Michael N. Helmus, Worcester, MA (US); Paul Valint, Pittsford, NY (US); Shrirang V. Ranade, Arlington, MA (US)

Correspondence Address: MAYER & WILLIAMS PC, 251 NORTH AVENUE WEST, 2ND FLOOR, WESTFIELD, NJ, 07090, US

(21) Appl. No.: 11/048,147

(22) Filed: Feb. 1, 2005

(51) Int. Cl. A61F 13/00

(52) U.S. Cl. 424/422

(57) ABSTRACT

An implantable or insertable medical device is provided that contains at least one polymeric region which comes into contact with a subject upon implantation or insertion of the device into the subject. The polymeric region(s) contain at least one bulk polymer moiety and at least one surface-active polymer moiety that (a) is covalently attached to the bulk polymer moiety/moieties or admixed with the bulk polymer moiety/moieties and (b) is provided in an amount that is effective in providing the polymeric region(s) with a critical surface energy that is between 20 dynes/cm and 30 dynes/cm upon implantation or insertion of the device into the subject.

### STATEMENT OF RELATED APPLICATION

This application is related to U.S. Ser. No. 10/830,772 filed Apr. 23, 2004 and entitled “Implantable or Insertable Medical Articles having Covalently Modified, Biocompatible Surfaces,” which is incorporated herein by reference in its entirety.

### TECHNICAL FIELD

This invention relates to implantable or insertable medical articles having biocompatible surfaces and to methods for providing the same.

### BACKGROUND

A wide variety of medical devices are known, which are adapted for implantation or insertion into the human body. Examples include catheters, cannulae, metal wire ligatures, stents, balloons, filters, scaffolding devices, coils, valves, grafts, plates, and other prosthesis which are adapted for implantation or insertion into various bodily locations, including the heart, coronary vasculature, peripheral vasculature, lungs, trachea, esophagus, intestines, stomach, brain, liver, kidney, bladder, urethra, ureters, eye, pancreas, ovary, and prostate. In many instances, such medical devices are equipped for the delivery of therapeutic agents. For example, an implantable or insertable medical device, such as a stent or a catheter, may be provided with a polymer matrix that contains a therapeutic agent. Once the medical device is placed at a desired location within a patient, the therapeutic agent is released from the polymer matrix and into the patient, thereby achieving a desired therapeutic outcome.

Regardless of whether or not the implantable or insertable medical device is adapted for release of a therapeutic agent, the surface regions of the medical device that come into contact with the body must be sufficiently biocompatible for the intended use of the device. The present invention is directed to the creation of medical devices having biocompatible surface regions.

### SUMMARY OF THE INVENTION

In accordance with an aspect of the present invention, an implantable or insertable medical device is provided that contains at least one polymeric region which comes into contact with a subject upon implantation or insertion of the device into the subject. The at least one polymeric region contains at least one bulk polymer moiety and at least one surface-active polymer moiety, which (a) is covalently attached to the bulk polymer moiety/moieties or admixed with the bulk polymer moiety/moieties and (b) is provided in an amount that is effective to provides the polymeric region(s) with a critical surface energy that is between 20 dynes/cm and 30 dynes/cm upon implantation or insertion of the device into the subject.

An advantage of the present invention is that novel medical devices are provided, which have a critical surface energy that has been shown to display enhanced biocompatibility, including enhanced throboresistance, relative to surfaces having other surface energies.

These and other embodiments and advantages of the present invention will become immediately apparent to those of ordinary skill in the art upon review of the Detailed Description and Claims to follow.

### BRIEF DESCRIPTION OF THE DRAWINGS

FIGS. 1A-1E are schematic illustrations of some polymer architectures in accordance with the present invention.

### DETAILED DESCRIPTION

The present invention is directed to implantable or insertable medical devices having biocompatible surfaces. In this regard, the medical devices of the present invention are provided with at least one polymeric region at their surfaces. The at least one polymeric region, in turn, contains at least one bulk polymer moiety and at least one surface-active polymer moiety that provides the polymeric region with a critical surface energy that is between 20 dynes/cm and 30 dynes/cm upon implantation or insertion of the device into a subject. The surface-active polymer moiety can be either admixed with the bulk polymer moiety/moieties or covalently attached to the bulk polymer moiety/moieties.

In some embodiments, the polymeric region corresponds to a coating that extends over all or a portion of a medical device substrate (e.g., where a medical device substrate, such as a metallic stent, is coated with a polymeric layer). In other embodiments, the polymeric region corresponds to a component of a medical device. In still other embodiments, the polymeric region corresponds to an entire medical device (e.g., where the polymeric region corresponds to a polymeric stent).

As used herein, “polymeric regions” are regions containing at least 50 wt % polymers, typically at least 75 wt %, at least 90 wt %, at least 95 wt %, or more, polymers.

“Polymers” and “polymer segments” are molecules and portions of molecules, respectively, which contain at least one polymer chain, which in turn contains multiple copies of one or more types of constituents, commonly called monomers. Polymer chains in accordance with the present invention contain 10 or more monomers, commonly 20 or more, 50 or more, 100 or more, 200 or more, 500 or more, or even 1000 or more monomers. An example of a common polymer is polystyrene, 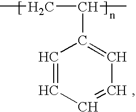
where n is an integer, typically an integer of 10 or more, more typically on the order of 10's, 100's, 1000's or even more, in which the constituents in the chain correspond to styrene: 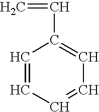
(i.e., they originate from, or have the appearance of originating from, the polymerization of styrene, in this case, the addition polymerization of styrene monomers).

A “constituent” is a portion of a molecule that that is not a polymer chain, although multiple constituents (i.e., monomers) may form a polymer chain.

A “segment” or “molecular segment” is a portion of a molecule, which may or may not contain one or more polymer chains. A “polymer segment” is a portion of a molecule, which contains one or more polymer chains, as noted above.

A “polymer moiety” is a molecule or a portion of a molecule, which contains one or more polymer chains.

“Bulk polymer moieties” are molecules or portions of molecules, other than the surface-active polymer moieties that provide the polymeric regions of the present invention with a critical surface energy that is between 20 dynes/cm and 30 dynes/cm upon implantation or insertion.

In certain embodiments, surface-active polymer moieties in accordance with the present invention contain the following: (a) at least one type of hydrophilic constituent (for example, the polymer moieties may be formed using a single type of hydrophilic monomer or other small molecule, or using a plurality of different hydrophilic monomer types or other small molecule types) and (b) at least one type of surface-energy-regulating constituent (for example, the polymer moieties may be formed using a single type of surface-energy-regulating monomer or other small molecule, or using a plurality of surface-energy-regulating monomer types or other small molecule types).

Being surface active, these polymer moieties concentrate at the surface of the polymeric region, maximizing their ability to influence the surface energy of the polymeric region. By providing suitable surface-active polymer moieties in suitable amounts, polymeric regions with a critical surface energy that is between 20 and 30 dynes/cm are created.

Surfaces having a critical surface energy between 20-30 dynes/cm have been shown in work by Dr. Robert Baier and others to provide enhanced biocompatibility, including enhanced thromboresistance. See, e.g., Baier R E, Meenaghan M A, Hartman L C, Wirth J E, Flynn H E, Meyer A E, Natiella J R, Carter J M, “Implant Surface Characteristics and Tissue Interaction”, J Oral Implantol, 1988, 13(4), 594-606; Robert Baier, Joseph Natiella, Anne Meyer, John Carter, “Importance of Implant Surface Preparation for Biomaterials with Different Intrinsic Properties in Tissue Integration in Oral and Maxillofacial Reconstruction”; Current Clinical Practice Series #29, 1986; Robert Baier, Joseph Natiella, Anne Meyer, John Carter, Fomalik, M. S., Tumbull, T., “Surface Phenomena in In Vivo Environments. Applications of Materials Sciences to the Practice of Implant Orthopedic Surgery”, NATO Advanced Study Institute, Costa Del Sol, Spain, 1984; Baier R E, Meyer A E, Natiella J R, Natiella R R, Carter J M, “Surface properties determine bioadhesive outcomes: methods and results”, JBiomed Mater Res, 1984, 18(4), 327-355; Joseph Natiella, Robert Baier, John Carter, Anne Meyer, Meenaghan, M. A., Flynn, H. E., “Differences in Host Tissue Reactions to Surface-Modified Dental Implants”, 185th ACS National Meeting, American Chemical Society, 1983.

Methods are known for measuring the critical surface energies of surfaces and include the use of contact angle methods to produce a Zisman Plot for calculating critical surface tensions as described in Zisman, W. A., “Relation of the equilibrium contact angle to liquid and solid constitution,” Adv. Chem. Ser. 43, 1964, pp. 1-51; Baier R. E., Shiafrin E. G., Zisman, W. A., “Adhesion: Mechanisms that assist or impede it,” Science, 162: 1360-1368, 1968; Fowkes, F. M., “Contact angle, wettability and adhesion,” Washington DC, Advances in Chemistry, vol. 43, 1964, p. 1, Souheng Wu, Polymer Interface and Adhesion, Marcel Dekker, 1982, Chapter 5, pp.169-212.

As indicated above, the critical surface energies of the polymeric regions of medical devices in accordance with the present invention are brought into the desired critical surface energy range of between 20 and 30 dynes/cm, by providing the polymeric regions with at least one surface-active polymer moiety. In certain embodiments, such surface-active polymer moieties contain, for example, (a) at least one type of hydrophilic constituent and (b) at least one type of surface-energy-regulating constituent.

In this regard, the effect of the surface-energy-regulating constituents is enhanced by concentrating these constituents at the surface of the device (which can occur either before, during or after insertion in the subject). This is done by further providing the surface-active polymer moieties with hydrophilic constituents that have an affinity for aqueous environments, such as the biological milieu that is present within the subject. The hydrophilic constituents will also commonly be repelled from the bulk of the polymeric region (e.g., due to hydrophobic-hydrophilic interactions). At the same time, care is taken to ensure that the surface-active polymer moieties have some affinity for the polymers forming the bulk of the polymeric regions, i.e., the bulk polymer moieties. This can be done, for example, by covalently attaching the surface-active polymer moieties to the bulk polymer moiety/moieties or by providing the surface-active polymer moieties as molecules, which are separate from the bulk polymer moiety/moieties, but which have an affinity for the bulk polymer moiety/moieties based on one or more physico-chemical forces such as electrostatic forces (e.g., charge-charge interactions, charge-dipole interactions, and dipole-dipole interactions, including hydrogen bonding), hydrophobic interactions, Van der Waals forces, and/or physical entanglements.

Consequently, the surface-active polymer moieties of the invention have a tendency to migrate to the surface of the polymeric region, enhancing their ability to alter the critical surface energy of the polymeric region to between 20 and 30 dynes/cm. As a result, the polymeric region is provided with an optimal surface energy for enhanced biocompatibility, including enhanced vascular compatibility. At the same time, because the surface-active polymer moieties also have an affinity toward the polymer(s) that form the bulk of the polymeric region, the surface-active polymer moieties remain associated with the medical device, rather departing into the surrounding biological environment, upon implantation or insertion.

Suitable hydrophilic constituents for use in forming the surface-active polymer moieties of the present invention can be selected, for example, from one or more of the following hydrophilic monomers: hydroxy-olefin monomers, such as vinyl alcohol and ethylene glycol; amino olefin monomers, such as vinyl amines; alkyl vinyl ether monomers, such as methyl vinyl ether; other hydrophilic vinyl monomers, such as vinyl pyrrolidone; methacrylic monomers, including methacrylic acid, methacrylic acid salts and methacrylic acid esters, for instance, alkylamino methacrylates and hydroxyalkyl methacrylates such as hydroxyethyl methacrylate; acrylic monomers such as acrylic acid, its anhydride and salt forms, and acrylic acid esters, for instance, hydroxyalkyl acrylates and alkylamino acrylates; cyclic ether monomers such as ethylene oxide; monosaccharides including aldoses such as glyceraldehyde, ribose, 2-deoxyribose, arabinose, xylose, glucose, mannose, and galactose, and ketoses such as ribulose, xylulose, fructose, and sorbose; nucleic acids; and amino acids.

In some embodiments, the surface-active polymer moieties will contain one or more distinct hydrophilic molecular segments. Suitable hydrophilic molecular segments can be selected, for example, from the following hydrophilic polymer segments: polysaccharide segments such as carboxymethyl cellulose and hydroxypropyl methylcellulose, polypeptide segments, poly(ethylene glycol) segments, poly(vinyl pyrrolidone) segments, poly(hydroxyethyl methacrylate) segments, and so forth. Hydrophilic polymer segments can be provided within the surface-active polymer moieties of the present invention in various configurations, for example, as polymer backbones, as polymer side chains, as polymer end groups, as polymer internal groups, and so forth.

In various embodiments, the hydrophilic molecular segments are selected from chemical entities that bind to proteins, cells and tissues within the biological milieu, and include, for example, hydrophilic polypeptide segments, hydrophilic polynucleotide segments, hydrophilic lipid segments (e.g., phospholipids segments), hydrophilic polysaccharide segments, hydrophilic antibody segments, and small-molecule segments, which can bind based, for example, on protein-protein interactions, protein-lipid interactions, protein-nucleic acid interactions, protein-polysaccharide interactions, protein-small molecule interactions, antibody-antigen interactions, nucleic acid-nucleic acid interactions, and so forth.

As noted previously, surface-active polymer moieties in accordance with the present invention are selected to ensure that the biological milieu is presented with a polymeric region that has a critical surface energy that is between 20 and 30 dynes/cm upon implantation or insertion of the device into a subject. To achieve this end, the surface-active polymer moieties in accordance with the present invention typically contain at least one type of surface-energy-regulating constituent in addition to the at least one type of hydrophilic constituent discussed above.

Examples of surface-energy-regulating constituents can be selected, for example, from the following: constituents that are rich in methyl groups, fluorocarbon constituents, alkyl methacrylate constituents, dialkylsiloxane constituents, hexatriacontane radicals, toluidine red radicals, and octadecylamine radicals.

In this connection, surface-active polymer moieties in accordance with the present invention can be provided with one or more polymer segments selected from the following: polymer segments that are rich in methyl groups, for example, polymer segments containing butyl acrylate monomers, such as poly (tert-butyl acrylate) segments, and polymer segments containing alkylene monomers, such as polyisobutylene segments; polymer segments formed from fluorocarbon monomers such as vinyl fluoride monomers, vinylidene fluoride monomers, monofluoroethylene monomers, 1,1-difluoroethylene monomers, trifluoroethylene monomers, and tetrafluoroethylene monomers, for example, polymer segments containing poly(vinyl fluoride), poly(vinylidene fluoride), poly(monofluoroethylene), poly(1,1 -difluoroethylene) or poly(trifluoroethylene), polymer segments containing a mixture of tetrafluoroethylene and chlorinated tetrafluoroethylene as monomers (e.g., in a 60/40 or in a 80/20 molar ratio), or polymer segments containing a mixture of ethylene and tetrafluoroethylene as monomers (e.g., in a 50/50 molar ratio); polymer segments containing alkyl methacrylate monomers, such as n-hexyl methacrylate monomers, octyl methacrylate monomers, lauryl methacrylate monomers, and stearyl methacrylate monomers, for instance, polymer segments containing poly(n-hexyl methacrylate), poly(octyl methacrylate), poly(lauryl methacrylate), or poly(stearyl methacrylate); and polymer segments containing dialkylsiloxane monomers such as poly(dimethylsiloxane). As with hydrophilic polymer segments, surface-energy-regulating polymer segments can be provided within the surface-active polymer moieties of the present invention in various configurations, for example, as polymer backbones, as polymer side chains, as polymer end groups, as polymer internal groups, and so forth.

For further information on critical surface energies of many of the above and various other materials, see, e.g., Arthur W. Adamson, Physical Chemistry of Surfaces, 3^{rd} ed., John Wiley, 1976, pg. 355; and Souheng Wu, Polymer Interface and Adhesion, Marcel Dekker, 1982, pp. 184-188.

It is beneficial in some embodiments to use a combination of surface-energy-regulating molecular segments to optimize surface properties, for instance, to reduce surface tack while at the same time maintaining the desired surface energy. For example, in one exemplary embodiment, the surface-active polymer moiety contains a combination of the following: (a) at least one surface-energy-regulating molecular segment such as poly(butyl acrylate), which may have the desired critical surface energy due to a high concentration of methyl groups, but which may also exhibit high tack, which is undesirable in some applications and (b) at least one surface-energy-regulating molecular segment, such as poly(monofluoroethylene), poly(1,1 -difluoroethylene) or poly(trifluoroethylene), which is should reduce the surface tack, while maintaining the desired surface energy.

In other embodiments, the surface-active polymer moieties of the present invention contain at least one surface-energy-regulating molecular segment that has a critical surface energy that is outside of the 20 to 30 dynes/cm range. However, when such surface-active polymer moieties are provided within the polymeric regions of the invention, along with the bulk polymer moieties, the critical surface energy of the polymeric regions are nevertheless brought within the 20 to 30 dynes/cm range.

For instance, in some embodiments, the surface-active polymer moieties contain surface-energy-regulating molecular segments with an energy below the desired 20 to 30 dynes/cm range, for example, in order to offset the presence of bulk polymer moieties within the polymeric regions which have surface energies above the 20 to 30 dynes/cm range, or to offset the presence of other molecular segments within the surface-active polymer moieties which have surface energies above the 20 to 30 dynes/cm range (e.g., high surface energy hydrophilic segments, such as polyethylene oxide segments). Conversely, in some embodiments, the surface-active polymer moieties contain surface-energy-regulating molecular segments with an energy above the desired 20 to 30 dynes/cm range, for example, in order to offset the presence of bulk polymer moieties within the polymeric regions which have surface energies below the 20 to 30 dynes/cm range, or to offset the presence of molecular segments within the surface-active polymer moieties which have surface energies below the 20 to 30 dynes/cm range.

Bulk polymer moieties for use in the polymeric regions of the present invention can be selected from a wide range of polymers, which may be homopolymers or copolymers (including alternating, random, statistical, gradient and block copolymers), which may be of cyclic, linear or branched architecture (e.g., the polymers may have star, comb or dendritic architecture), which may be natural or synthetic, and so forth. Suitable bulk polymer moieties may be selected, for example, from the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides and polyether block amides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinyl acetate copolymers (EVA), polyvinylidene chlorides, polyvinyl ethers such as polyvinyl methyl ethers; vinyl aromatic polymers and copolymers such as polystyrenes, styrene-maleic anhydride copolymers, vinyl-aromatic-olefin copolymers including styrene-butadiene copolymers, styrene-ethylene-butylene copolymers (e.g., a polystyrene-polyethylene/butylene-polystyrene (SEBS) copolymer, available as Kratong® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene block copolymers such as those disclosed in U.S. Pat. No. 6,545,097 to Pinchuk et al.), polyvinyl ketones, polyvinylcarbazoles, and polyvinyl esters such as polyvinyl acetates; polybenzimidazoles; ethylene-methacrylic acid copolymers and ethylene-acrylic acid copolymers, where some of the acid groups can be neutralized with either zinc or sodium ions (commonly known as ionomers); polyalkyl oxide polymers and copolymers including polyethylene oxides (PEO); polyesters including polyethylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof; examples of commercially available polyurethane copolymers include Bionate®, Carbothane®, Tecoflex®, Tecothane®, Tecophilic®, Tecoplast®, Pellethane®, Chronothane® and Chronoflex®); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as derivatives, and additional blends and copolymers of the above.

In some embodiments, the surface-active polymer moieties of the present invention are provided with one or more polymer segments, which have constituents that match those found within the bulk polymer moieties of the polymeric regions, thereby enhancing the interaction between the surface-active polymer moieties and the bulk polymer moieties.

As with other polymers and polymer segments described herein, surface-active polymer moieties can have a near-infinite variety of architectures, including cyclic, linear and branched architectures. Branched architectures include star-shaped architectures (e.g., architectures in which three or more chains emanate from a single branch point), comb architectures (e.g., architectures having a main chain and a plurality of side chains), dendritic architectures (e.g., arborescent and hyperbranched polymers), and so forth.

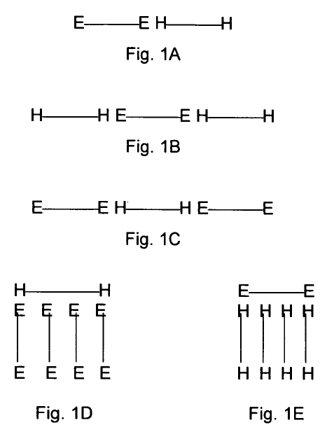

A few specific examples of surface-active polymer moiety architectures are illustrated schematically in FIGS. 1A-1E. In these specific examples, hydrophilic polymer segments are denoted by H-H , while surface-energy regulating polymer segments are denoted by E-E. If present, linking regions are not illustrated.

FIG. 1A illustrates a simple linear diblock copolymer, whereas FIGS. 1B-1C illustrate triblock copolymers, each having a “two-arm” configuration. Although not illustrated, three-arm, four-arm, etc. configurations can be constructed by selecting a multi-functional center segment. FIGS. 1D-1E, on the other hand, illustrate “comb” or “graft” configurations, each having multiple side chains. For instance, in FIG. 1D, a plurality of surface-energy regulating polymer segments emanate as side chains from a hydrophilic polymer backbone segment, whereas in FIG. 1E a plurality of hydrophilic polymer segments emanate as side chains from a surface-energy regulating polymer backbone segment.

Although the hydrophilic and surface-energy regulating constituents are provided in distinct polymer segments in the examples of FIG. 1A-1E, in other instances these constituents are intermixed. For example hydrophilic and surface-energy regulating monomers can be intermixed in a periodic (e.g., alternating), random, statistical, or gradient fashion, as described below.

A wide variety of techniques, including various polymerization and grafting techniques are known, which can be employed in the construction of the surface-active polymer moieties of the present invention.

Specific examples of surface-active polymer moieties in accordance with the invention include copolymers of hydrophilic (meth)acrylate monomers and alkyl(meth)acrylate monomers (note that the parenthetical “meth” in the term “(meth)acrylate” is optional; thus “alkyl(meth)acrylate” is a shorthand notation that embraces both “alkyl acrylate” and “alkyl methacrylate”). The molecule 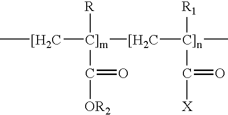
one example, where R is hydrogen or methyl, R₁ is hydrogen or methyl, R₂ is a linear, branched or cyclic alkyl group containing from 1 to 18 carbons and is selected to provide the resulting copolymer with the desired surface energy modifying characteristics, and X is a branched or unbranched hydroxyalkyl group having from 1 to 4 carbons and from 1 to 4 hydroxyl groups (e.g., a hydroxyethyl group, a hydroxypropyl group, a dihydroxypropyl group) or an alkylamino group containing 1 or 2 branched or unbranched alkyl groups having 1 to 4 carbons (e.g., an N,N-dimethylamino group). The number of alkyl(meth)acrylate monomers and hydrophilic (meth)acrylate monomers, m and n, typically range, independently, from 10 to 5000, and can be provided within the copolymer in any order. For example, the copolymer can be a block copolymer, a periodic (e.g., alternating) copolymer, a random copolymer, a statistical copolymer, a gradient copolymer, and so forth. (A diblock copolymer will take on the appearance of FIG. 1A).

Other specific examples of surface-active polymer moieties in accordance with the invention include copolymers having hydrophilic side chains and surface-energy-regulating backbone segments, for instance, copolymers which are formed by the copolymerization of a methoxypoly(oxyethylene)methacrylate macromonomer (or “macromer”) with a hydrophobic monomer such as an alkyl(meth)acrylate monomer, in which the alkyl group is selected to provide the resulting copolymer with the desired surface energy modifying characteristics. Conversely, specific examples of copolymers having surface-energy-regulating side chains and hydrophilic backbone segments include those which are formed by the copolymerization of a mono-methacrylated-polyalkyl(meth)acrylate macromer with a hydrophilic monomer such as hydroxyethylmethacrylate or N,N-dimethylacrylamide.

In view of the above, it should be clear to one of ordinary skill in the art that a wide range of surface-active polymer moieties may be formed using a wide variety of polymerization and/or linking chemistries that are known in the polymerization art.

As discussed above, in addition to the at least one surface-active polymer moiety, the polymeric regions of the present invention also contain at least one bulk polymer moiety. The surface-active polymer moieties of the present invention can be associated with the bulk polymer moieties in various ways. For example, in some embodiments, surface-active polymer moieties are provided, which contain reactive groups that allow them to be covalently attached to the bulk polymer moieties. In other embodiments, the surface-active polymer moieties contain constituents that have an affinity for the bulk polymer moiety (e.g., surface-energy-regulating constituents, in some cases, or other constituents which are supplied for purposes of promoting interaction with the bulk polymer moiety). In either case, the surface-active polymer moieties will tend to move to the interface with the biological milieu, while at the same time remaining anchored to the bulk polymer moiety.

In some cases, the implantable or insertable medical devices of the invention are further provided with a therapeutic agent, for example, by providing the therapeutic agent within or beneath the polymeric regions. Where utilized, the therapeutic agent is introduced into the medical devices before or after the formation of the polymeric regions. For example, in certain embodiments, the therapeutic agent is formed concurrently with the polymeric region. In other embodiments, the therapeutic agent is dissolved or dispersed within a solvent, and the resulting solution contacted with a previously formed polymeric region to incorporate the therapeutic agent into the polymeric region. In still other embodiments the polymeric region is formed or adhered over a region that comprises the therapeutic agent.

Therapeutic agents are provided in accordance with the present invention for any of a number of purposes, for example, to effect in vivo release (which may be, for example, immediate or sustained) of the biologically active agents, to affect tissue adhesion vis-à-vis the medical device, to influence thromboresistance, to influence antihyperplastic behavior, to enhance recellularization, and to promote tissue neogenesis, among many other purposes.

Medical devices for use in conjunction with the present invention include those that are implanted or inserted into the body and can be selected, for example, from the following: orthopedic prosthesis such as bone grafts, bone plates, joint prosthesis, central venous catheters, vascular access ports, cannulae, metal wire ligatures, stents (including coronary vascular stents, cerebral, urethral, ureteral, biliary, tracheal, gastrointestinal and esophageal stents), stent grafts (e.g., endovascular stent-grafts), vascular grafts, catheters (for example, renal or vascular catheters such as balloon catheters), guide wires, balloons, filters (e.g., vena cava filters), tissue scaffolding devices, tissue bulking devices, embolization devices including cerebral aneurysm filler coils (e.g., Guglilmi detachable coils, coated metal coils and various other neuroradiological aneurysm coils), heart valves, left ventricular assist hearts and pumps, artificial heart housings, and total artificial hearts.

The medical devices of the present invention may be used for essentially any therapeutic purpose, including systemic treatment or localized treatment of any mammalian tissue or organ. Examples include tumors; organs including but not limited to the heart, coronary and peripheral vascular system (referred to overall as “the vasculature”), lungs, trachea, esophagus, brain, liver, kidney, bladder, urethra and ureters, eye, intestines, stomach, pancreas, ovary, and prostate; skeletal muscle; smooth muscle; breast; cartilage; and bone. As used herein, “treatment” refers to the prevention of a disease or condition, the reduction or elimination of symptoms associated with a disease or condition, or the substantial or complete elimination of a disease or condition. Typical subjects (also referred to as “patients”) are vertebrate subjects, more typically mammalian subjects and even more typically human subjects.

Numerous techniques are available for forming the polymeric regions of the invention, including thermoplastic and solvent based techniques. For example, where polymer species forming the polymeric regions (e.g., the surface-active polymer moiety and bulk polymer moiety, which may be attached or unattached to the surface-active polymer moiety) have thermoplastic characteristics, a variety of standard thermoplastic processing techniques can be used to form the same, including compression molding, injection molding, blow molding, spinning, vacuum forming and calendaring, as well as extrusion into sheets, fibers, rods, tubes and other cross-sectional profiles of various lengths. Using these and other techniques, entire devices or portions thereof can be made. For example, an entire stent can be extruded using the above techniques. As another example, a coating can be provided by extruding a coating layer onto a pre-existing stent. As yet another example, a coating can be co-extruded with an underlying stent body. If a therapeutic agent is to be provided, and it is stable at processing temperatures, then it can be combined with the polymer(s) prior to thermoplastic processing. If not, then is can be added to a preexisting polymer region.

When using solvent-based techniques, the surface-active polymer moiety and bulk polymer moiety (which may be attached or unattached to the surface-active polymer moiety) are typically first dissolved or dispersed in a solvent system and the resulting mixture is subsequently used to form the polymeric region. The solvent system that is selected will typically contain one or more solvent species. Preferred solvent-based techniques include, but are not limited to, solvent casting techniques, spin coating techniques, web coating techniques, solvent spraying techniques, dipping techniques, techniques involving coating via mechanical suspension including air suspension, ink jet techniques, electrostatic techniques, and combinations of these processes.

In certain embodiments, a mixture containing solvent, surface-active polymer moiety and bulk polymer moiety (which may be attached or unattached to the surface-active polymer moiety), as well as any optional supplemental species and/or therapeutic agent, is applied to a substrate to form a polymeric region. For example, the substrate can be all or a portion of an underlying support material (e.g., a metallic, polymeric or ceramic implantable or insertable medical device or device portion, such as a stent) to which the polymeric region is applied. On the other hand, the substrate can also be, for example, a removable substrate, such as a mold or another template, from which the polymeric region is separated after solvent elimination. In still other techniques, for example, fiber forming techniques, the polymeric region is formed without the aid of a substrate.

Although various embodiments are specifically illustrated and described herein, it will be appreciated that modifications and variations of the present invention are covered by the above teachings and are within the purview of the appended claims without departing from the spirit and intended scope of the invention.

1. An implantable or insertable medical device comprising a polymeric region which comes into contact with a subject upon implantation or insertion of the device into the subject, said polymeric region comprising a bulk polymer moiety and a surface-active polymer moiety that is (a) covalently attached to the bulk polymer moiety or admixed with the bulk polymer moiety and (b) supplied in an amount that is effective to provide said polymeric region with a critical surface energy that is between 20 dynes/cm and 30 dynes/cm upon implantation or insertion of said device into the subject.

2. The implantable or insertable medical device of claim 1, comprising a plurality of said polymeric regions.

3. The implantable or insertable medical device of claim 1, wherein said surface-active polymer moiety comprises hydrophilic and surface-energy-regulating constituents.

4. The implantable or insertable medical device of claim 3, wherein said surface-active polymer moiety comprises hydrophilic and surface-energy-regulating monomers.

5. The implantable or insertable medical device of claim 4, wherein said hydrophilic monomers are selected from hydroxy-olefin monomers, amino olefin monomers, alkyl vinyl ether monomers, vinyl pyrrolidone, methacrylic acid, methacrylic acid salts, alkylamino methacrylate monomers, hydroxyalkyl methacrylate monomers; acrylic acid, acrylic acid salts, alkylamino acrylate monomers, hydroxyalkyl acrylate monomers, and cyclic ether monomers.

6. The implantable or insertable medical device of claim 4, wherein said surface-energy-regulating monomers are selected from fluorocarbon monomers, alkyl methacrylate monomers, dialkylsiloxane monomers.

7. The implantable or insertable medical device of claim 4, wherein said hydrophilic and surface-energy-regulating monomers are arranged in a random, statistical, gradient or periodic distribution in one or more polymer segments within said surface-active polymer moiety.

8. The implantable or insertable medical device of claim 4, wherein said surface-active polymer moiety comprises a surface-energy-regulating polymer segment comprising said surface-energy-regulating monomer and a hydrophilic polymer segment comprising said hydrophilic monomer.

9. The implantable or insertable medical device of claim 1, wherein said surface-active polymer moiety comprises a hydrophilic polymer segment selected from a poly(hydroxy-olefin) segment, a poly(amino-olefin) segment, a poly(alkyl vinyl ether) segment, a poly(vinyl pyrrolidone) segment, a poly(hydroxyalkyl acrylate) segment, a poly(hydroxyalkyl methacrylate) segment, a poly(alkylamino acrylate) segment, a poly(alkylamino methacrylate) segment, a poly(ethylene oxide) segment, a polysaccharide segment, a polynucleotide segment and a polypeptide segment.

10. The implantable or insertable medical device of claim 1, wherein said surface-active polymer moiety comprises a 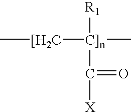
segment, where n is an integer from 10 to 5000, R₁ is hydrogen or methyl, X is a branched or unbranched hydroxyalkyl group having from 1 to 4 carbons and having from 1 to 4 hydroxyl groups or an alkylamino group comprising from 1 to 2 branched or unbranched alkyl groups and having from 1 to 4 carbons.

11. The implantable or insertable medical device of claim 1, wherein said surface-active polymer moiety comprises a hydrophilic polymer segment selected from a poly(ethylene glycol) segment, a poly(vinyl pyrrolidone) segment, a carboxymethyl cellulose segment, a hydroxypropyl methylcellulose segment, and a poly(hydroxyethyl methacrylate) segment.

12. The implantable or insertable medical device of claim 1, wherein said surface-active polymer moiety comprises a plurality of hydrophilic polymer segments.

13. The implantable or insertable medical device of claim 12, wherein at least one of said plurality of hydrophilic polymer segments comprises a monomeric constituent that is not found in at least one other of said plurality of hydrophilic polymer segments.

14. The implantable or insertable medical device of claim 3, wherein said surface-active polymer moiety comprises a phospholipid as a hydrophilic constituent.

15. The implantable or insertable medical device of claim 1, wherein said surface-active polymer moiety comprises a surface-energy-regulating polymer segment.

16. The implantable or insertable medical device of claim 15, wherein said surface-energy-regulating polymer segment is selected from a poly(butyl acrylate) segment, a poly(vinyl fluoride) segment, a poly(vinylidene fluoride) segment, a poly(monofluoroethylene) segment, a poly(1,1 -difluoroethylene) segment, a poly(trifluoroethylene) segment, a poly(n-hexyl methacrylate) segment, a poly(octyl methacrylate) segment, a poly(lauryl methacrylate) segment, a poly(stearyl methacrylate) segment, a poly(dimethylsiloxane) segment, a copolymer segment comprising tetrafluoroethylene and chlorinated tetrafluoroethylene, and a copolymer segment comprising ethylene and tetrafluoroethylene.

17. The implantable or insertable medical device of claim 1, wherein said surface-active polymer moiety comprises a plurality of surface-energy-regulating polymer segments.

18. The implantable or insertable medical device of claim 17, wherein at least one of said plurality of surface-energy-regulating polymer segments comprises a monomeric constituent that is not found in at least one other of said plurality of surface-energy-regulating polymer segments.

19. The implantable or insertable medical device of claim 1, wherein said surface-active polymer moiety is covalently attached to said bulk polymer moiety.

20. The implantable or insertable medical device of claim 1, wherein said surface-active polymer moiety is admixed with said bulk polymer moiety.

21. The implantable or insertable medical device of claim 20, wherein said surface-active polymer moiety further comprises a polymer segment that has an affinity for said bulk polymer moiety.

22. The implantable or insertable medical device of claim 20, wherein said polymer segment is selected from a polyacrylate segment, a polymethacrylate segment, a polyurethane segment, polyolefin segment, poly(vinyl aromatic) segment, and a silicone segment.

23. The implantable or insertable medical device of claim 15, wherein said surface-active polymer moiety comprises a 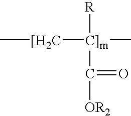
segment, where m is an integer ranging from 10 to 5000, R is hydrogen or methyl, and R₂ is a linear, branched or cyclic alkyl group containing from 1 to 18 carbons.

24. The implantable or insertable medical device of claim 1, wherein said bulk polymer moiety is a homopolymer or block copolymer, said bulk polymer moiety comprising a polymer segment selected from a polyacrylate segment, a polymethacrylate segment, a polyurethane segment, a polyolefin segment, a poly(vinyl aromatic) segment, and a silicone segment.

25. The implantable or insertable medical device of claim 1, further comprising a therapeutic agent dispersed or dissolved within said polymeric region.

26. The implantable or insertable medical device of claim 1, wherein said polymeric region is in the form of a polymeric coating disposed over an underlying substrate.

27. The implantable or insertable medical device of claim 1, wherein said medical device is selected from vascular stents, vascular catheters, prosthetic heart valves, artificial heart housings, vascular grafts, endovascular stent-grafts, and neuroradiological aneurysm coils.
